(19) United States
(12) Patent Application Publication
(10) Pub. No.: US 2005/0119587 A1
(43) Pub. Date: Jun. 2, 2005

(54) Method and apparatus for evaluating connective tissue conditions

(75) Inventors: Blake Roessler, Ann Arbor, MI (US); Michael D. Morris, Ann Arbor, MI (US); Steven A. Goldstein, Ann Arbor, MI (US); Abigail Smukler, Ann Arbor, MI (US); Nicole Crane, Ypsilanti, MI (US); Barbara R. McCreadie, Ann Arbor, MI (US); Tso-Ching Chen, Whitmore Lake, MI (US)

Correspondence Address: MARSHALL, GERSTEIN & BORUN LLP, 6300 SEARS TOWER, 233 S. WACKER DRIVE, CHICAGO, IL, 60606, US

(73) Assignee: UNIVERSITY OF MICHIGAN, Ann Arbor, MI (US)

(21) Appl. No.: 10/944,518

(22) Filed: Sep. 17, 2004

Related U.S. Application Data
(63) Continuation-in-part of application No. 10/879,797, filed on Jun. 29, 2004.
(60) Provisional application No. 60/484,198, filed on Jul. 1, 2003.

(51) Int. Cl. A61B 10/00

(52) U.S. Cl. 600/562

(57) ABSTRACT

In a method for evaluating a connective tissue condition of a patient, an indicator of the supporting tissue condition may be generated. A portion of connective tissue of the patient is irradiated using a light source. The connective tissue may be irradiated in vivo through the skin or via an incision, for example. Alternatively, a biopsy of the connective tissue may be irradiated. Then, spectral content information for light scattered, reflected, or transmitted by the connective tissue is determined. The spectral content information is used, at least in part, to generate the indicator. The indicator may assist a physician in diagnosing or ruling out the connective tissue condition, determining a risk of developing a disease, monitoring the progression of a disease, monitoring a response to treatment, etc.

### CROSS-REFERENCE TO RELATED APPLICATIONS

The present application is a continuation-in-part of U.S. patent application Ser. No. 10/879,797, entitled “Method and Apparatus for Diagnosing Bone Tissue Conditions,” filed on Jun. 29, 2004, which claims the benefit of U.S. Provisional Application No. 60/484,198, filed Jul. 1, 2003. Both of these applications are hereby incorporated by reference herein in their entireties for all purposes.

### STATEMENT REGARDING FEDERALLY SPONSORED RESEARCH OR DEVELOPMENT

This invention was made with Government support under Grant numbers P30 AR46024, R01 AR34399, and R01 AR47969 awarded by the Public Health Service division of the Department of Health and Human Services. The Government may own certain rights in this invention.

### FIELD OF THE DISCLOSURE

The present disclosure generally relates to medical diagnostic apparatus and methods, and more particularly to apparatus and methods that may be used to help diagnose conditions of connective tissue.

### BACKGROUND

Osteoporosis is an important healthcare problem. It is estimated that 24 million Americans are affected by osteoporosis and that osteoporosis led to $13.8 billion in healthcare costs in 1995. The risk of dying from hip fracture complications is the same as the risk of dying from breast cancer. For Caucasian females over 50, the risk of hip, spine, or distal forearm fractures is 40%. Osteoporosis is currently defined as a condition in which bone mineral density is greater than two standard deviations below the mean of a young healthy population.

Current techniques for screening individuals for fracture susceptibility are relatively inaccurate and/or pose risks to the patient. For example, the present preferred technique for diagnosis of osteoporosis is dual X-ray absorption (DXA), which measures the amount of mineral in the bone. In some patients, however, a low mineral content does not appear to lead to an increased risk of fracture. Additionally, DXA requires that the patient is exposed to ionizing radiation.

Osteoarthritis is another important health care problem. It has been estimated that 40 million Americans and 70 to 90 percent of persons older than 75 years are affected by osteoarthritis. The prevalence of osteoarthritis among men and women is equal, though its symptoms occur earlier in women. Risk factors include age, joint injury, obesity, and mechanical stress.

Studies suggest physio-chemical alteration of the articular cartilage surface is an early event in the pathogenesis of osteoarthritis. The changes involve physical damage to structural matrix proteins, mediated by physical forces and degradative enzymes.

Current techniques for diagnosing or ruling out osteoarthritis include taking an X-ray image of a joint, analyzing blood samples, and analyzing synovial fluid withdrawn from the joint with a needle. The diagnosis is largely clinical because radiographic findings do not always correlate with symptoms. An X-ray image of a joint may indicate osteoarthritis if a normal space between the bones in a joint is narrowed, an abnormal increase in bone density is evident, or if bony projections or erosions are evident. A blood sample may indicate osteoarthritis if byproducts of hyaluronic acid are present. Hyaluronic acid is a joint lubricant and the presence of its byproducts in the blood may indicate the lubricant's breakdown, a sign of osteoarthritis. Also, elevated levels of a factor called C-reactive protein, which is produced by the liver in response to inflammation, may indicate osteoarthritis. On the other hand, elevated levels of rheumatoid factor and so-called erythrocyte sedimentation rates may indicate rheumatoid arthritis rather than osteoarthritis. An analysis of synovial fluid withdrawn from the joint may indicate osteoarthritis if cartilage cells are present in the fluid. On the other hand, a high white blood cell count in the synovial fluid is an indication of infection, and high uric acid in the synovial fluid is an indication of gout.

### SUMMARY

Methods and apparatus are provided for evaluating a connective tissue condition of a patient (e.g., a disease, a risk of developing a disease, a risk of developing a fracture, etc.). For example, an indicator associated with the supporting tissue condition may be generated. First, a portion of connective tissue of the patient is irradiated using a light source. The connective tissue may be irradiated in vivo through the skin or via an incision, for example. Alternatively, a biopsy of the connective tissue may be irradiated. Then, spectral content information for light scattered, reflected, or transmitted by the connective tissue is determined. The spectral content information is used, at least in part, to generate the indicator. The indicator may assist a physician in diagnosing or ruling out the connective tissue condition. Also, the indicator may assist in estimating a risk of fracture, estimating a risk of developing a connective tissue disease, monitoring the progression of a connective tissue disease, monitoring a response to treatment of a connective tissue disease, etc.

In one embodiment, an apparatus is provided that includes a light source, and a light receiver to receive light from a portion of connective tissue of a patient irradiated by the light source. Additionally, a spectrum analyzer is optically coupled to receive light received by the light receiver. Further, a computing device is communicatively coupled to the spectrum analyzer and is configured to generate diagnostic information indicative of the connective tissue condition based at least in part on spectral content information.

In another aspect, a method for determining whether a patient has a cartilage tissue condition is provided. The method includes irradiating a portion of cartilage tissue of the patient using a light source, and receiving light from the portion of the cartilage tissue. The method also includes determining Raman spectra information associated with the received light, and generating, based at least on the Raman spectra information, an indicator of the cartilage tissue condition.

In yet another embodiment, apparatus for evaluating a cartilage tissue condition comprises a light source and a Raman probe to receive light scattered from a portion of cartilage tissue of a patient irradiated by the light source. The apparatus also comprises a spectrum analyzer coupled to receive light received by the light receiver and to determine Raman spectra information for the received light. The apparatus further comprises a computing device coupled to the spectrum analyzer, the computing device configured to generate diagnostic information indicative of the cartilage tissue condition based at least in part on the Raman spectra information.

### BRIEF DESCRIPTION OF THE DRAWINGS

The features and advantages of the apparatus and methods described herein will be best appreciated upon reference to the following detailed description and the accompanying drawings, in which:

FIG. 1 is a block diagram of one embodiment of an apparatus for determining susceptibility to fracture;

FIG. 2 is a flow diagram of one embodiment of a method for determining a susceptibility to fracture;

FIG. 3 is a flow diagram of one embodiment of a method for determining a susceptibility to fracture based on spectral content information;

FIG. 4 is a flow diagram of another embodiment of a method for determining a susceptibility to fracture based on spectral content information;

FIG. 5 is a chart showing measured spectral content information for a group of patients that suffered fractures and for a control group;

FIG. 6 is a block diagram of a computer that can be used with the apparatus of FIG. 1;

FIG. 7 is a flow diagram of one embodiment of a method for determining a cartilage tissue condition;

FIG. 8 is a flow diagram of one embodiment of a method for determining a cartilage tissue condition based on spectral content information;

FIG. 9 is a flow diagram of another embodiment of a method for determining a cartilage tissue condition based on spectral content information;

FIG. 10 is a flow diagram of another embodiment of a method for determining a cartilage tissue condition based on spectral content information;

FIG. 11A is a chart showing measured spectral content information associated with cartilage tissue for wildtype mice; and

FIG. 11B a chart showing measured spectral content information associated with cartilage tissue for transgenic mice.

### DETAILED DESCRIPTION

Diagnostic Apparatus

FIG. 1 is a block diagram of an example apparatus 100 that may be used to help diagnose a condition of the bone tissue of a patient. For example, the apparatus 100 may be used to help diagnose osteoporosis, help estimate a susceptibility to fracture of the bone tissue, help diagnose a defect (e.g., osteogenesis imperfecta), help diagnose a nutritional disorder, or help diagnose other disorders related to bone tissue. The apparatus 100 may be used on a patient once, for example, or may be used multiple times, over time to help track changes in the bone tissue.

The apparatus 100, which may be used for a Raman spectrometry analysis of a bone tissue or an infrared (IR) analysis of the bone tissue, includes a light source 104 optically coupled to at least one optical fiber 108. For Raman spectrometry, the light source 104 may comprise a laser, for example, that generates substantially monochromatic light. The optical fiber 108 is optically coupled to an optical probe 116. The optical probe 116 may be positioned proximate to a portion of bone tissue 120 from a patient, and may be used to irradiate the bone tissue 120 with the light generated by the light source 104.

In one embodiment, the optical probe 116 is also optically coupled to at least another optical fiber 124. In this embodiment, the optical probe 116 may be used to collect light scattered or reflected by the bone tissue 120 and to transmit the scattered light through the optical fiber 124. This embodiment may be used for Raman spectrometry or for “attenuated total reflection” IR spectrometry.

In another embodiment, another optical probe 128 may be positioned proximate to the portion of the bone tissue 120 such that the optical probe 128 can collect light transmitted by the bone tissue 120. The optical probe 128 may be optically coupled to the optical fiber 124 and can transmit the light transmitted by the bone tissue 120 through the optical fiber 124. This embodiment may be used for “line of sight” IR spectrometry.

The optical fiber 124 is optically coupled to a spectrum analyzer 132 via an optical processor 140 which may include one or more lenses and/or one or more filters. The spectrum analyzer 132 may include, for example, a spectrograph optically coupled to an array of optical detectors, and is communicatively coupled to a computing device 144.

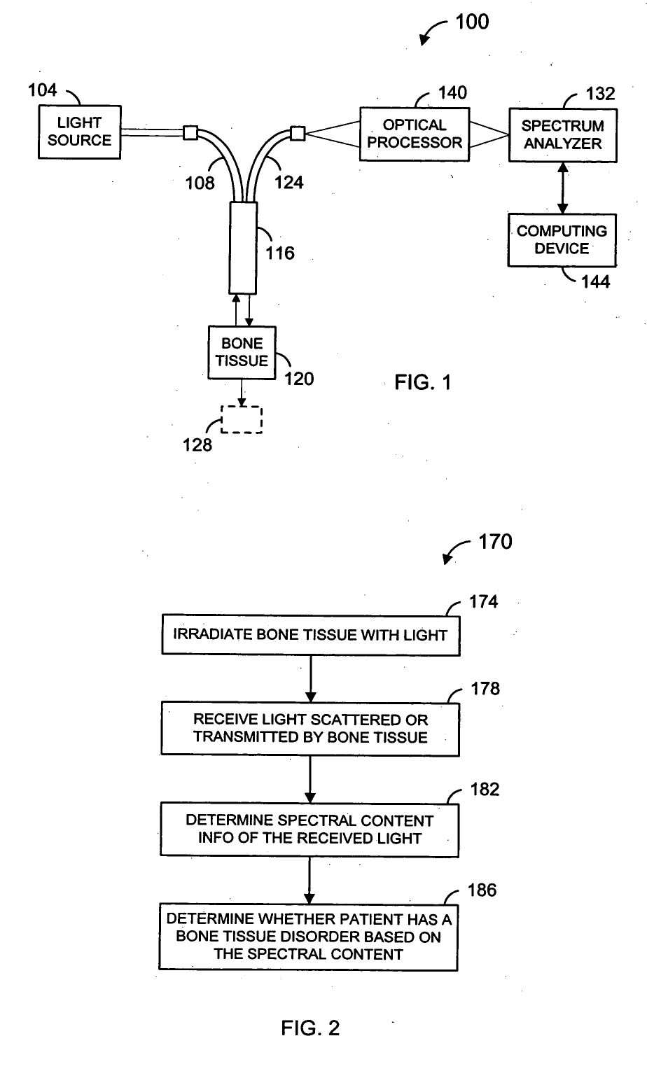

FIG. 2 is a flow diagram of a method for determining a condition related to the bone tissue of a patient. The method 170 may be implemented by an apparatus such as the apparatus 100 of FIG. 1, and will be described with reference to FIG. 1. At a block 174, a portion of bone tissue of a patient is irradiated with light. For example, the optical probe 116 may be used to irradiate the bone tissue 120 with light generated by the light source 104. In one embodiment, the bone tissue 120 may be irradiated non-invasively through the skin of the patient. In other embodiments, bone tissue 120 exposed by an incision, or removed as a biopsy, may be irradiated.

In some embodiments, bone tissue at or near a site presumed at risk for fracture (e.g., the hip) may be irradiated. Alternatively, bone tissue not at or near a site of presumed risk may be measured. For in vivo measurements, irradiation may occur at a site at which bone tissue is close to the skin. For example, the proximal diaphysis of the tibia may be irradiated. As biopsy measurements, an iliac crest biopsy could be irradiated as just one., example.

At a block 178, light scattered, reflected, or transmitted by the bone tissue may be collected. For example, the optical probe 116 may collect light scattered by the bone tissue 120 (Raman spectrometry). As another example, the optical probe 116 may collect light reflected by the bone tissue 120 (“attenuated total reflection” IR spectrometry). Alternatively, the optical probe 128 may collect light transmitted by the bone tissue 120 (“line of sight” IR spectrometry). As with the optical probe 116, the optical probe 128 may collect light non-invasively through the skin of the patient. In other embodiments, the light may be collected via an incision or collected from an irradiated biopsy.

At a block 182, spectral content information associated with the collected light is generated. For example, the light collected by the optical probe 116 or the optical probe 128 may be provided to the spectrum analyzer 132 via the optical processor 140. The spectrum analyzer 132 may then generate spectral content information associated with the light received by the spectrum analyzer 132.

In Raman spectrometry the collected light may include light at wavelengths shifted from the wavelength of the incident light the spectrum of the collected light scattered from bone tissue (referred to hereinafter as the “Raman spectrum of the bone tissue”) is indicative of the physico-chemical state of the bone tissue. The Raman spectrum of the bone tissue includes bands indicative of various components of the bone tissue including phosphate of bone mineral, carbonate of bone mineral, interstial water, residual water, hydroxide of the bone mineral, etc. Also included are bands indicative of various components of the collagen matrix of the bone tissue including amide I, hydroxyproline, proline, cross-links, etc. The wavelength at which a band is located is indicative of the component of the bone mineral or matrix to which it corresponds. The height and/or intensity of a band is indicative of the amount of the corresponding component of the bone tissue.

In IR spectrometry, the light generated by the light source 104 includes light at a variety of IR wavelengths. Some of the light at various wavelengths is absorbed by components of the bone tissue, and different components absorb different wavelengths. Thus, similar to the Raman spectrum of the bone tissue, in IR spectrometry, the spectrum of the collected light transmitted by the bone tissue (referred to hereinafter as the “IR spectrum of the bone tissue”) includes bands indicative of components and structure of the bone tissue. Unlike in Raman spectrometry, however, the bands in the IR spectrum of the bone tissue are indicative of light absorbed by the bone tissue, rather than light scattered by the bone tissue. Nevertheless, the IR spectrum of the bone tissue is also indicative of the physico-chemical state of the bone tissue. As is known to those of ordinary skill in the art, the Raman spectrum of a bone tissue and an IR spectrum of the same bone tissue may provide indications of different components and/or different structure of the bone tissue.

At a block 186, it is determined whether the patient has a bone tissue disorder based on the spectral content information generated at block 182. For example, the computing device 144 may receive spectral content information from the spectrum analyzer 132. The computing device 144 may then generate an indication of whether the patient has a bone tissue disorder. As another example, the computing device 144 may generate an indication, based on the spectral content information generated at block the 182, that may be used by a physician to determine whether the patient has a bone tissue disorder. For example, the indication may be indicative of a susceptibility of the bone tissue of the patient to fracture. The bone tissue disorder may be, for example, osteoporosis, a genetic disorder (e.g., osteogenesis imperfecta), an acquired disorder, etc.

The determination of the block 186 may be based on additional factors. For example, the determination may be further based on one or more of an age of the patient, a height of the patient, a weight of the patient, a bone mineral density of the patient (e.g., determined using DXA), a family history of the patient, etc. Determining the estimate of susceptibility to fracture will be described in more detail below.

Blocks 174, 178, and 182 may optionally be repeated over a period of time (e.g, weeks, months, years) to generate spectral content information that reflects the condition of the bone tissue of the patient over the period of time. This spectral content information over the period of time may be used in the determination of block 186.

Estimating Susceptibility to Fracture

In one embodiment, the determination of block 186 comprises estimating a susceptibility of the bone tissue of the patient to fracture. Examples of techniques for estimating a susceptibility to fracture based on spectral content information are provided below. Many other techniques may be employed as well. In general, embodiments of methods for estimating susceptibility to fracture may vary according to the environment in which they are to be used. For example, different embodiments may be used in a clinical setting as compared to a laboratory setting because signal-to-noise ratios likely will be higher in the laboratory setting as compared to the clinical setting.

In some embodiments in which Raman spectrometry is employed, the area under a band or height of particular bands in the Raman spectrum of the bone tissue may be used to determine a susceptibility to fracture.

Amide I and amide III are observable in both IR and Raman spectrometry. Amide I and amide III spectra include information similarly indicative of the structure of collagen in the bone tissue, although amide I appears to produce more intense bands as compared to amide III. In Raman spectrometry, amide I of bone tissue is associated with a plurality of bands that can extend over much of the 1600 cm⁻¹ to 1700 cm⁻¹ region. For example, amide I of bone tissue is associated with a band approximately at 1650 cm⁻¹ and a band approximately at 1680 cm⁻¹ to 1690 cm⁻¹.

It is believed that the absence of collagen intrafibral cross-links weakens bone tissue. The disruption or absence of collagen cross-links can result in changes to the relative intensities of the bands associated with amide I. For example, denaturing collagen to gelatin causes the high frequency shoulder associated with amide I to become more prominent. Additionally, the intrafibril cross-links in bone matrix collagen cause shifts in the proline bands (proline-2 and proline-3) from 1660 cm⁻¹ to 1663 cm⁻¹ and from 1670 cm⁻¹ to 1690 cm⁻¹ respectively. Research has shown that the 1690 cm⁻¹ band intensity in bone matrix increases relative to the intensity of the 1663 cm⁻¹ band when dehydrodihydroxylysinonorleucine, dehydrohydroxylysinonorleucine or dehydrohistindohydroxymerodesmosine cross-links are chemically reduced. Further research with fetal murine calvarial tissue has shown that the matrix amide I band in newly deposited tissue has a prominent shoulder at approximately 1690 cm⁻¹ that becomes smaller as the tissue ages and cross-links are formed.

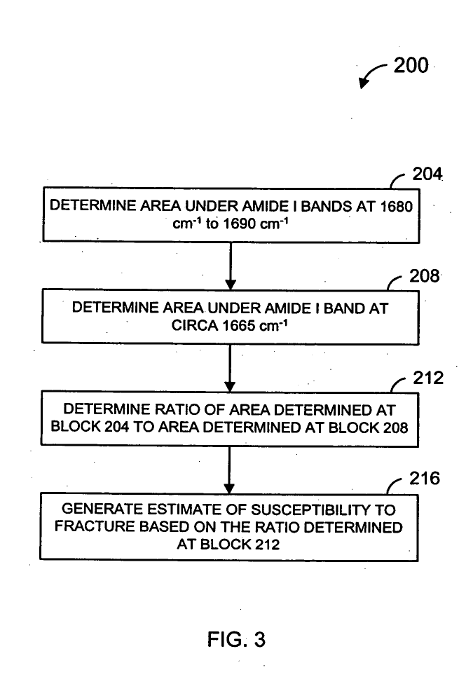

FIG. 3 is a flow diagram illustrating one embodiment of a method for determining susceptibility to fracture based on areas of particular bands in a Raman spectrum of bone tissue. A similar technique may be employed for use with an IR spectrum of bone tissue.

At a block 204, an area of the amide I bands substantially between 1680 cm⁻¹ and 1690 cm⁻¹ is determined. Determining the area of these amide I bands may include curve fitting using a function such as a mixed Gaussian-Lorentzian function. Determining the area of the bands may also include measuring the area without curve fitting. For example, the area could be measured based on the raw data. As another example, the raw data could be filtered (e.g., with a smoothing filter), and the area could be measured based on the filtered data. In general, the areas under one or more bands may be determined using any of a variety of techniques, including known techniques. At a block 208, an area of the amide I band approximately at 1665 cm⁻¹ is determined. Determining the area of this amide I band may be performed in the same or similar manner as described with reference to block 204.

At a block 212, a ratio of the area determined at the block 204 with the area determined at the block 208 may be determined. Then, at a block 216, an estimate of the susceptibility to fracture of the bone tissue is determined based on the ratio determined at the block 212. Determining the estimate of the susceptibility to fracture may comprise determining in which of one or more sets of values the ratio falls. In one embodiment, the estimate of the susceptibility to fracture may comprise an indication of whether or not the bone tissue is susceptible to fracture. In other embodiments, the estimate of the susceptibility to fracture may additionally comprise an indication of one of a plurality of risk levels (e.g., high risk, increased risk, normal risk).

As described previously the estimate of the susceptibility to fracture determined at the block 216 may be based on additional factors such as one or more of an age of the patient, a height of the patient, a weight of the patient, a bone mineral density of the patient, a family history of the patient, etc.

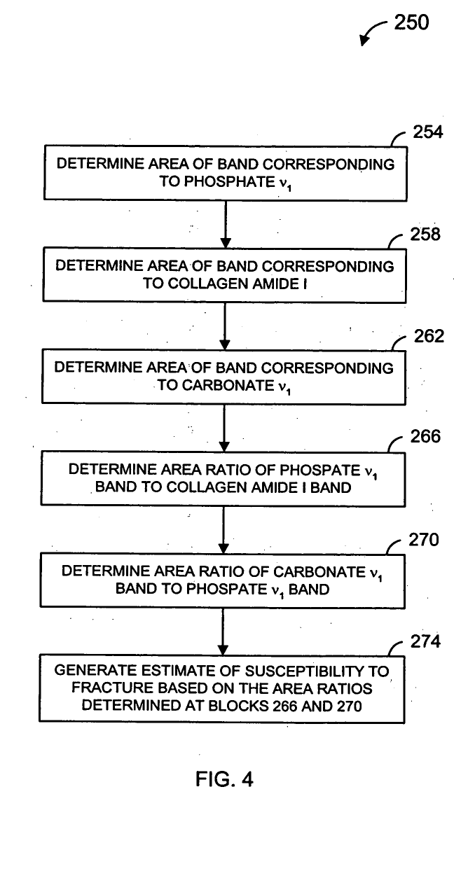

FIG. 4 is a flow diagram illustrating another embodiment of a method for determining susceptibility to fracture based on areas of particular bands. At a block 254, an area of a band associated with phosphate ν₁ and having a peak at approximately 957 cm⁻¹ and having a shoulder at approximately 945 cm⁻¹ is determined. Other phosphate bands could be used, although it is believed that the ν₁ band is more intense than other phosphate bands. Determining the area of this phosphate ν₁ band may include curve fitting to resolve the phosphate ν₁ band into two components using a function such as a mixed Gaussian-Lorentzian function or some other suitable function. In general, the area of this band may be performed using any of a variety of techniques, including known techniques such as those described previously. ν1

At a block 258, the area of the collagen amide I envelope (the plurality of bands between approximately 1600 cm⁻¹ to 1700 cm⁻¹) is determined. Other matrix bands could be used, for example bands indicative of hydroxyproline (853 cm⁻¹), proline (919 cm⁻¹), etc. Determining the area of the collagen amide I band may be performed in the same or similar manner as described previously. At a block 262, the area of the carbonate ν₁ band (circa 1070 cm⁻¹) is determined. Determining the area of the carbonate ν₁ band may be performed in the same or similar manner as described previously. Additionally, other carbonate bands could be used, although it is believed that the ν₁ band is more intense than other carbonate bands.

At a block 266, a ratio of the area of the phosphate ν₁ band to the area of the collagen amide I bands is determined. At a block 270, a ratio of the area of the carbonate ν₁ band to the area of phosphate ν₁ band is determined. It is believed that this ratio is a rough measure of the size and crystallinity of mineral crystals.

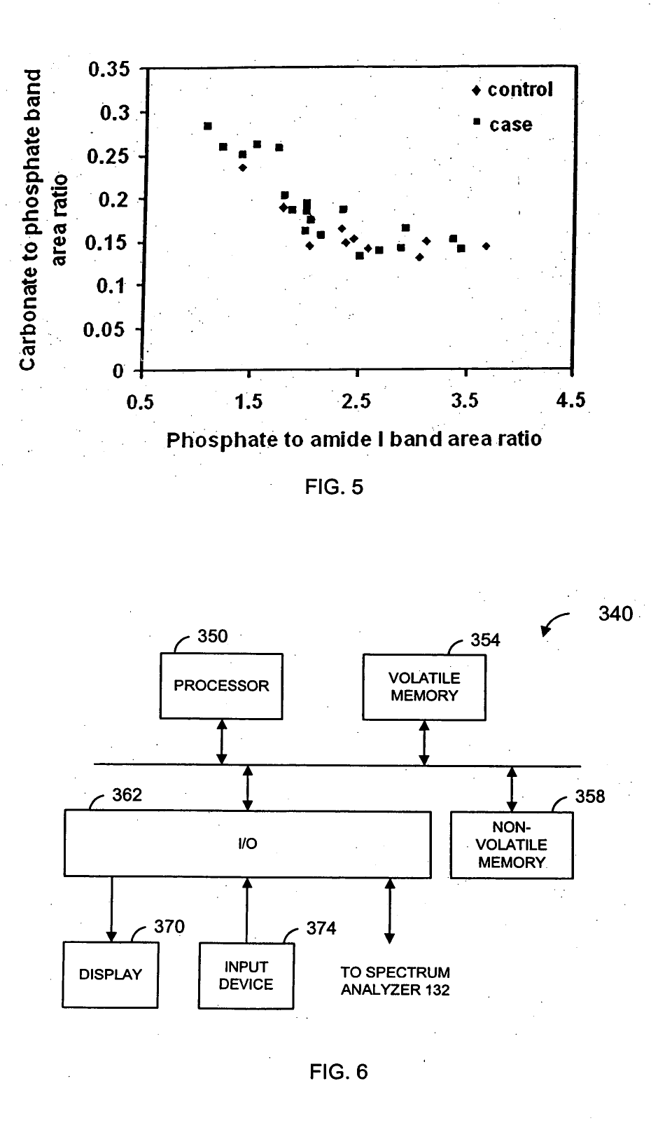

FIG. 5 is a plot of the above-described ratios determined from bone tissue taken from the proximal femur in the same location for each individual in a matched set of females. A control group included eleven individuals who had died without having a hip fracture. A fracture group included eighteen individuals who had sustained a hip fracture and were treated with arthroplasty. In the fracture group, those who had sustained fracture due to trauma such as automobile accidents or falls from a ladder were excluded. The control group and the fracture group were selected such that the age of the individuals and the bone volume fractions were similar between the two groups.

As can be seen in FIG. 5, a relationship exists between the carbonate/phosphate ratio and the phosphate/amide I ratio. As the phosphate/amide I ratio decreases, the carbonate/phosphate ratio at first generally remains approximately constant. As the phosphate/amide I ratio continues to decrease, the carbonate/phosphate ratio then tends to increase considerably. The fracture specimens tend to be concentrated at the low end of the phosphate/amide I ratio range, while the control specimens tend to be concentrated at the upper end of the phosphate/amide I ratio range. A low phosphate/amide I ratio and a high carbonate/phosphate ratio appear strongly associated with hip fracture. Student t-tests were conducted on the data illustrated graphically in FIG. 4. A comparison of the carbonate/phosphate ratios between the two groups (the fracture group and the control group) resulted in a p-value of 0.08. A comparison of the phosphate/collagen ratios between'the two groups resulted in a p-value of 0.28.

Referring again to FIG. 4, at a block 274, an estimate of the susceptibility to fracture of the bone tissue is determined based on the ratios determined at the blocks 266 and 270. Determining an estimate of the susceptibility to fracture may comprise determining whether the ratios determined at the blocks 266 and 270 fall within one or more sets of values. Additionally, in one embodiment, the estimate of the susceptibility to fracture may comprise an indication of whether or not the bone tissue is susceptible to fracture. In other embodiments, the estimate of the susceptibility to fracture may additionally comprise an indication of one of a plurality of risk levels (e.g., high risk, increased risk, normal risk).

The estimate of the susceptibility to fracture determined at the block 274 may be based on additional factors such as one or more of an age of the patient, a height of the patient, a weight of the patient, a bone mineral density of the patient, a family history of the patient, etc. Additionally, the estimate of the susceptibility to fracture determined at block 274 may be based on spectral content information taken over a period of time (e.g., weeks, months, years).

Other information in the IR spectrum or the Raman spectrum of the bone tissue can be used in addition to, or as an alternative, the information described above. For example, information related to bands other than those described above could be used. Additionally, information related to the width, shape (e.g., whether or not a band has “shoulders”), height, etc. of particular bands could be used in determining susceptibility to fracture. Additionally, more sophisticated analyses could be employed such as a cluster analysis.

In a study separate from the study associated with the data of FIG. 5, iliac crest biopsies were analyzed from ten subjects without fractures (mean age 56 years, range 43-70 years) and five subjects with osteoporotic fractures (mean age 63 years, range 50-72 years). In particular, for each specimen, trabecular and cortical regions were scanned using Raman spectroscopy and average carbonate/phosphate and phosphate/amide I band area rations were obtained for the trabecular and cortical regions. No corrections were made for multiple comparisions.

Both carbonate ν₁/phosphate ν₁ ratio and phosphate ν₁/amide I ratio were higher in cortical than trabecular bone for all specimens (p=0.005 and p=0.01, respectively, paired t-tests). This may suggest that mineralized matrix chemistry differs between bone types due to, for example, a fundamental difference or a result of differing average tissue age. Chemical composition of cortical bone mineralized matrix appears to change with age, as demonstrated by a decrease in phosphate/amide I ratio (p=0.005, linear regression model). Neither carbonate ν₁/phosphate ν₁ ratio in cortical bone nor any measure in trabecular bone showed significant change with age. The phosphate ν₁/amide I ratio in patients without fractures was greater in cortical than trabecular bone until age 55 (in all 6 subjects), but greater in trabecular bone in those 55 y or older (in all 4 subjects). In all 5 patients with fractures, the phosphate ν₁/amide I ratio was greater in cortical bone. Thus, patients with fractures demonstrated the pattern seen in younger (under 55) non-fractured subjects, as opposed to the pattern of patients of similar age without fractures. It is possible that failure to alter mineralized matrix chemistry results in increased fracture risk. Another possibility is that the greater-phosphate ν₁/amide I ratio in cortical bone for patients with fractures, as compared to phosphate ν₁/amide I ratio in the trabecular bone, was a result of the fracture. There may be other explanations as well for the differences in the relationship between phosphate ν₁/amide I ratio in cortical bone and trabecular bone between patients with fractures and patients without fractures.

Comparing patients with fractures- to patients without fractures, trabecular bone from patients with fractures had a lower phosphate ν₁/amide I ratio (p=0.03, t-test). No differences appeared to be found in cortical bone or in carbonate ν₁/phosphate ν₁ ratio in trabecular bone. This lower mineral/matrix ratio (decreased mineral) in trabecular bone with patients with fractures may suggest a systemic increase in remodeling prior to or following fracture, and is likely demonstrated more clearly in trabecular bone because of its more rapid turnover. If this increase in remodeling occurs prior to fracture, chemical composition from iliac crest biopsy specimens may improve fracture risk assessment. The lower phosphate ν₁/amide I ratio in trabecular bone for patients with fractures, however, could be a result of the fracture. There may be other explanations as well for the lower phosphate ν₁/amide I ratio in trabecular bone for patients with fractures.

Yet another study was conducted that was designed to help understand whether, and how, the chemical composition of the bone extracellular matrix changes immediately after fracture. Raman spectroscopy was used to compare chemical composition between the fracture site and a location away from the fracture site. With this experimental model, it was assumed that there was originally no difference along the length of the bone. It was also assumed that there was little change far from the fracture site as a result of the fracture. Thus, differences in chemical composition found in this study between the fracture site and far from it may model changes in the chemical composition of the bone as a result of the fractures.

In this study, the tibiae of five mice were fractured in a controlled manner. One day later, the tibiae were dissected out and Raman spectra were obtained for cortical bone at/near the fracture site and approximately 2 mm from the fracture site (no trabecular bone was analyzed). Data from both locations were available for 4 limbs, each from separate animals.

The results indicated a decreased phosphate ν₁/amide I ratio and increased carbonate ν₁/phosphate ν₁ ratio at the fracture site as compared to the site 2 mm away from the fracture. This data may suggest there is some change in the chemical composition of the bone extracellular matrix following fracture. It is important to note, however, that this assumes that there was no difference in chemical composition existed prior to the fracture between the two sites. It also assumes that there was little change at the site 2 mm away from the fracture site as a result of the fracture. There may be other explanations for why the study indicates decreased phosphate ν₁/amide I ratio and increased carbonate ν₁/phosphate ν₁ ratio at the fracture site as compared to the site 2 mm away from the fracture.

Further Description of the Diagnosis Apparatus

In general, embodiments of apparatus for determining a bone tissue disorder may vary in design according to the environment in which they are to be used. For example, an apparatus to be used in a clinical setting may be designed to obtain spectrum information more quickly as compared to an apparatus to be used in a laboratory setting.

Referring again to FIG. 1, many types of light sources 104 may be employed. With regard to Raman spectrometry, a substantially monochromatic light source can be used. In general, near-infrared wavelengths provide better depth of penetration into tissue. On the, other hand, as wavelengths increase, they begin to fall outside the response range of silicon photo detectors (which have much better signal-to-noise ratios than other currently available, detectors). One example of a light source that can be used is the widely available 830 nanometer diode laser. This wavelength can penetrate at least 1 to 2 millimeters into tissue. Additionally, this wavelength is not absorbed by blood hemoglobin and is only weakly absorbed by melanin. If the bone tissue is to be exposed by incision, or if biopsied bone tissue is to be examined, other wavelengths may be employed. For example, a 785 nanometer diode laser could be used.

Many other wavelengths may be used as well. In general, a wavelength of a light source may be chosen based on various factors including one or more of a desired depth of penetration, availability of photo detectors capable of detecting light at and near the wavelength, efficiency of photo detectors, cost, manufacturability, lifetime, stability, scattering efficiency, penetration depth, etc. Any of a variety of substantially monochromatic light sources can be used, including commercially available light sources. For example, the article “Near-infrared multichannel Raman spectroscopy toward real-time in vivo cancer diagnosis,” by S. Kaminaka, et al. (Journal of Raman Spectroscopy, vol. 33, pp. 498-502, 2002) describes using a 1064 nanometer wavelength light source with an InP/InGaAsP photomultiplier.

With regard to IR spectrometry, any of a variety of types of light sources can be used, including commercially available light sources. For example, light sources known to those of ordinary skill in the art as being suitable for analysis of bone tissues can be used.

With regard to the optical probe 116, any of variety optical probes can be used, including commercially available optical probes. For instance, the Handbook of Vibrational Spectroscopy, Volume 2: Sampling Techniques, 1587-1597 (J. Chalmers et al. eds., John Wiley & Sons Ltd. 2002) describes examples of fiber optic probes that can be used. For Raman spectrometry, optical probes designed for Raman spectrometry may be used. For example; any of a variety of commercially available fiber optic probes can be used. Some commercially available fiber optic probes include filters to reject Raman scatter generated within the excitation fiber and/or to attenuate laser light entering the collection fiber or fibers. Loosely focused light may help eliminate or minimize patient discomfort as compared to tightly focused light. As is known to those of ordinary skill in the art, loosely focused light may be achieved by a variety of techniques including multimode delivery fibers and a long focal length excitation/collection lens(es).

Existing commercially available fiber optic probes may be modified, or new probes developed, to maximize collection efficiency of light originating at depths of 1 millimeter or more below the surface of a highly scattering medium, such as tissue. Such modified, or newly developed probes, may offer better signal-to-noise ratios and/or faster data collection. The probe may be modified or may be coupled to another device to help maintain a constant probe-to-tissue distance, which may help to keep the system in focus and help maximize the collected signal.

If the bone is to be irradiated via an incision (and/or the light is to be collected via an incision), relay optics may be coupled to, or incorporated in, a needle. For example, two optical fibers or an “n-around-one” array could be used. In general, the size and the number of fibers should be appropriate to fit into a needle. The diameter of the excitation/collection lens or lenses used in such an embodiment could be small to help minimize the size of the incision. For example, lenses of diameters between 0.3 and 1 millimeter could be used. Lenses having larger or smaller diameters could be used as well. The lens(es) and or optical fibers could be incorporated into a hypodermic needle such as a #12 French type needle.

Additionally, a microprobe or microscope (e.g., a modified epi-fluorescence microscope) may be used instead of the optical probe 116 of FIG. 1. In this embodiment, the optical fiber 108 and/or the optical fiber 124 may be omitted.

The optical processor 140 may include one or more lenses for focusing the collected light. The optical processor 140 may also include one or more filters to attenuate laser light. Although shown separate from the spectrum analyzer 132, some or all of the optical processor 140 may optionally be a component of the spectrum analyzer 132.

The spectrum analyzer 132 may comprise a spectrograph optically coupled with a photo detector array. The photo detector array may comprise a charge coupled device, or some other photo detection device. For example, the article “Near-infrared multichannel Raman spectroscopy toward real-time in vivo cancer diagnosis,” by S. Kaminaka, et al. (Journal of Raman Spectroscopy, vol. 33, pp. 498-502, 2002) describes using a 1064 nanometer wavelength light source with an InP/InGaAsP photomultiplier.

In another embodiment, the spectrum analyzer 132 may comprise one or more filters to isolate a plurality of wavelengths of interest. Then, one or more photo detectors (e.g., a CCD, an avalanche photodiode, photomultiplier tube, etc.) could be optically coupled to the output of each filter. A single detector could be used with a tunable filter (e.g., an interferometer, liquid crystal tunable filter, acousto-optic tunable filter, etc.) or if fixed passband filters (e.g., dielectric filters, holographic filters, etc.) are placed in front of the detector one at a time using, for example, a slider, filter wheel, etc. In general, any of a variety of spectrum analyzers could be used such as a Raman analyzer, an IR spectrum analyzer, an interferometer, etc.

The computing device 144 may comprise, for example, an analog circuit, a digital circuit, a mixed analog and digital circuit, a processor with associated memory, a desktop computer, a laptop computer, a tablet PC, a personal digital assistant, a workstation, a server, a mainframe, etc. The computing device 144 may be communicatively coupled to the spectrum analyzer 132 via a wired connection (e.g., wires, a cable, a wired local area network (LAN), etc.) or a wireless connection (a BLUETOOTH™ link, a wireless LAN, an IR link, etc.). In some embodiments, the spectral content information generated by the spectrum analyzer 132 may be stored on a disk (e.g., a floppy disk, a compact disk (CD), etc.), and then transferred to the computing device 144 via the disk. Although the spectrum analyzer 132 and the computer 144 are illustrated in FIG. 1 as separate devices, in some embodiments the spectrum analyzer 132 and the computing device 144 may be part of a single device. For example, the computing device 144 (e.g., a circuit, a processor and memory, etc.) may be a component of the spectrum analyzer 132.

FIG. 5 is a block diagram of an example computing device 144 that may be employed. It is to be understood that the computer 340 illustrated in FIG. 5 is merely one example of a computing device 144 that may be employed. As described above, many other types of computing devices 144 may be used as well. The computer 340 may include at least one processor 350, a volatile memory 354, and a non-volatile memory 358. The volatile memory 354 may include, for example, a random access memory (RAM). The non-volatile memory 358 may include, for example, one or more of a hard disk, a read only memory (ROM), a CD-ROM, an erasable programmable ROM (EPROM), an electrically erasable programmable ROM (EEPROM), a digital versatile disk (DVD), a flash memory, etc. The computer 340 may also include an I/O device 362. The processor 350, volatile memory 354 non-volatile memory 358, and the I/O device 362 may be interconnected via one or more address/data buses 366. The computer 340 may also include at least one display 370 and at least one user input device 374. The user input device 374 may include, for example, one or more of a keyboard, a keypad, a mouse, a touch screen, etc. In some embodiments, one or more of the volatile memory 354, nonvolatile memory 358, and the I/O device 362 may be coupled to the processor 350 via one or more separate address/data buses (not shown) and/or separate interface devices (not shown), coupled directly to the processor 350, etc.

The display 370 and the user input device 374 are coupled with the I/O device 362. The computer 340 may be coupled to the spectrum analyzer 132 (FIG. 1) via the I/O device 362. Although the I/O device 362 is illustrated in FIG. 5 as one device, it may comprise several devices. Additionally, in some embodiments, one or more of the display 370, the user input device 374, and the spectrum analyzer 132 may be coupled directly to the address/data bus 366 or the processor 350. Additionally, as described previously, in some embodiments the spectrum analyzer 132 and the computer 340 may be incorporated into a single device.

The previously described additional factors that may be used for diagnosing a bone tissue disorder (e.g., one or more of an age of the patient, a height of the patient, a weight of the patient, a bone mineral density of the patient, a family history of the patient, etc.) may be entered via the user input device 374, loaded from a disk, received via a network (not shown), etc. These additional factors may be stored in one or more of the memories 354 and 358. Additionally, previously measured spectral content information may be loaded from a disk, received via a network (not shown), etc. and stored in one or more of the memories 354 and 358.

A routine, for example, for estimating a susceptibility to fracture based on spectral content information may be stored, for example, in whole or in part, in the non-volatile memory 358 and executed, in whole or in part, by the processor 350. For example, the method 200 of FIG. 3 and/or the method 250 of FIG. 4 could be implemented in whole or in part via a software program for execution by the processor 350. The program may be embodied in software stored on a tangible medium such as CD-ROM, a floppy disk, a hard drive, a DVD, or a memory associated with the processor 350, but persons of ordinary skill in the art will readily appreciate that the entire program or parts thereof could alternatively be executed by a device other than a processor, and/or embodied in firmware and/or dedicated hardware in a well known manner. With regard to the method 200 of FIG. 3 and the method 250 of FIG. 4, one of ordinary skill in the art will recognize that the order of execution of the blocks may be changed, and/or the blocks may be changed, eliminated, or combined.

Although the method 200 of FIG. 3 and the method 250 of FIG. 4 were described above as being implemented by the computer 340, one or more of the blocks of FIGS. 3 and 4 may be implemented by other types of devices such as an analog circuit, a digital circuit, a mixed analog and digital circuit, a processor with associated memory, etc.

Determining Cartilage Conditions

Although the example apparatus described above were described in the context of analyzing bone tissue, these apparatus or similar apparatus can be used to determine conditions associated with other connective tissues. Generally, connective tissue comprises a biological tissue having an extensive extracellular matrix. Connective tissue helps form a framework and/or support structure for body tissues, organs, etc. Examples of connective tissue that can be analyzed include supporting connective tissue (e.g., bone, cartilage, etc.), fibrous connective tissue (e.g., cartilage, tendons, ligaments, etc.), loose connective tissue, adipose tissue, etc.

As described above, connective tissues such as cartilage may be analyzed. At least some spectral information associated with cartilage can be distinguished from spectral information associated with bone, and thus techniques for determining cartilage conditions based on spectral information may be performed in vivo.

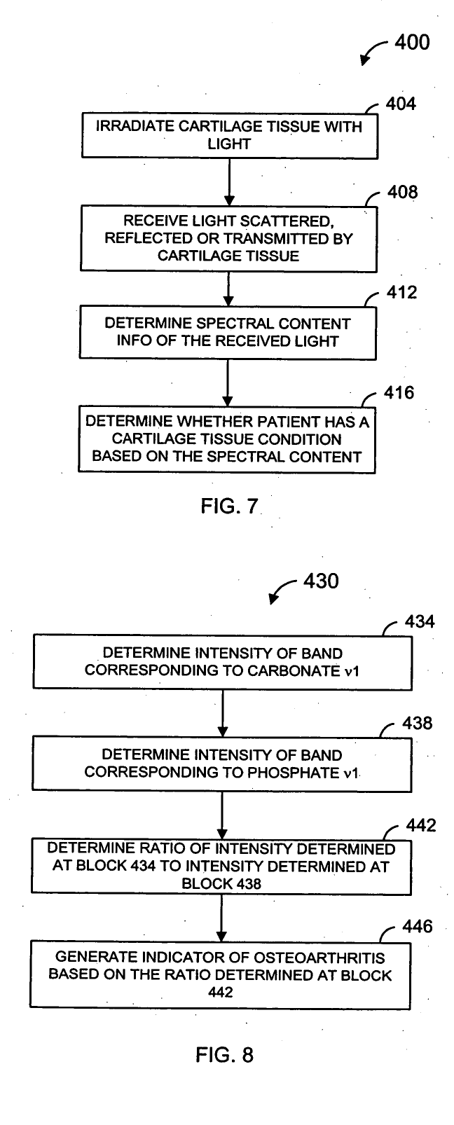

FIG. 7 is a flow diagram of an example method for determining a condition related to cartilage tissue of a patient. The method 400 may be implemented by an apparatus such as the apparatus 100 of FIG. 1, and will be described with reference to FIG. 1. At a block 404, a portion of cartilage tissue of a patient is irradiated with light. For example, the optical probe 116 may be used to irradiate the cartilage tissue with light generated by the light source 104. In one embodiment, the cartilage tissue may be irradiated non-invasively through the skin of the patient. In other embodiments, cartilage tissue exposed by an incision, or removed as a biopsy, may be irradiated.

At a block 408, light scattered, reflected, or transmitted by the cartilage tissue may be collected. For example, the optical probe 116 may collect light scattered by the cartilage tissue (Raman spectrometry). As another example, the optical probe 116 may collect light reflected by the cartilage tissue (“attenuated total reflection” IR spectrometry). Alternatively, the optical probe 128 may collect light transmitted by the cartilage tissue (“line of sight” IR spectrometry). As with the optical probe 116, the optical probe 128 may collect light non-invasively through the skin of the patient. In other embodiments, the light may be collected via an incision or collected from an irradiated biopsy.

At a block 412, spectral content information associated with the collected light is generated. For example, the light collected by the optical probe 116 or the optical probe 128 may be provided to the spectrum analyzer 132 via the optical processor 140. The spectrum analyzer 132 may then generate spectral content information associated with the light received by the spectrum analyzer 132.

In Raman spectrometry, the cartilage spectrum of the collected light scattered from cartilage tissue (referred to hereinafter as the “Raman spectrum of the cartilage tissue”) is indicative of the physico-chemical state of the cartilage tissue. The Raman spectrum of the cartilage tissue includes bands indicative of various components present in cartilage tissue including phosphate, carbonate, etc. Also included are bands indicative of various components of the collagen matrix of the cartilage tissue including amide I, amide III, etc. The wavelength at which a band is located is indicative of the component of the mineral or matrix to which it corresponds. The height and/or intensity of a band is indicative of the amount of the corresponding component.

Similar to the Raman spectrum of the cartilage tissue, in IR spectrometry, the spectrum of the collected light transmitted by the cartilage tissue (referred to hereinafter as the “IR spectrum of the cartilage tissue”) includes bands indicative of components and structure of the cartilage tissue. Unlike in Raman spectrometry, however, the bands in the IR spectrum of the cartilage tissue are indicative of light absorbed by the cartilage tissue, rather than light scattered by the cartilage tissue. Nevertheless, the IR spectrum of the cartilage tissue is also indicative of the physico-chemical state of the cartilage tissue. As is known to those of ordinary skill in the art, the Raman spectrum of a cartilage tissue and an IR spectrum of the same cartilage tissue may provide indications of different components and/or different structure of the cartilage tissue.

At a block 416, it is determined whether the patient has a cartilage tissue condition based on the spectral content information generated at block 412. For example, the computing device 144 may receive spectral content information from the spectrum analyzer 132. The computing device 144 may then generate an indication, based at least in part on, the spectral content information, of whether the patient has a cartilage tissue condition. The cartilage tissue condition may be, for example, osteoarthritis, rheumatoid arthritis, chondromalacia, polychondritis, relapsing polychondritis, a genetic disorder, an acquired disorder, etc. Also, the cartilage tissue condition may be an increased risk of developing a disease such as osteoarthritis, rheumatoid arthritis, chondromalacia, polychondritis, etc. A computing device such as the computing device 340 of FIG. 6 may be used to generate the indication. In some embodiments, the computing device 144 may generate, based on the spectral content information generated at block the 412, an indicator associated with the cartilage tissue condition. Such an indicator may be used by a physician to determine whether the patient has a cartilage tissue condition, to monitor the progression of a cartilage tissue condition, to monitor a response to treatment of a cartilage tissue condition, etc.

The determination of the block 416 may be based on additional factors. For example, the determination may be further based on one or more of an age of the patient, a weight of the patient, a history of weight of the patient, a blood test, an analysis of synovial fluid, a medical history of the patient (e.g., past joint injuries), an X-ray, a family history of the patient, etc. Determining whether the patient has a cartilage tissue condition will be described in more detail below.

Blocks 404, 408, and 412 may optionally be repeated over a period of time (e.g, weeks, months, years) to generate spectral content information that reflects the cartilage tissue condition of the patient over the period of time. This spectral content information over the period of time may be used in the determination of block 416.

Evaluating Osteoarthritis Condition

Examples of techniques for generating an indicator, based on spectral content information, of osteoarthritis are provided below. Many other techniques may be employed as well. In general, embodiments of methods for generating such an indicator may vary according to the environment in which they are to be used. For example, different embodiments may be used in a clinical setting as compared to a laboratory setting because signal-to-noise ratios likely will be higher in the laboratory setting as compared to the clinical setting.

In some embodiments in which Raman spectrometry is employed, the intensity of particular bands in the Raman spectrum of the cartilage tissue may be used to, generate an indicator of osteoarthritis. The intensity of a band may be determined by, for example, determining an area under the band or determining a height of the band.

Amide I and amide III are observable in both IR and Raman spectrometry. Amide I and amide III spectra include information similarly indicative of the structure of collagen in the cartilage tissue. In Raman spectrometry, amide III of cartilage tissue is associated with a plurality of bands that can extend over much of the 1240 cm⁻¹ to 1270 cm⁻¹ region. Also observable are bands associated with minerals present in the cartilage tissue. For example, bands associated with carbonate ν₁ and phosphate ν₁ are observable.

FIG. 8 is a flow diagram illustrating one embodiment of a method 430 for generating an indicator of a cartilage tissue condition based on intensities of particular bands in a Raman spectrum of cartilage tissue. A similar technique may be employed for use with an IR spectrum of cartilage tissue.

At a block 434, an intensity of a carbonate ν₁ band (nominally located at approximately 1070 cm⁻¹) associated with the cartilage tissue is determined. Determining the intensity of this band may include measuring the height of a peak of the band. Also, determining the intensity of this band may include determining the area under the band by curve fitting using a function such as a mixed Gaussian-Lorentzian function. Determining the area of the band may also include measuring the area without curve fitting. For example, the area could be measured based on the raw data. As another example, the raw data could be filtered (e.g., with a smoothing fitter), and the height or area could be measured based on, the filtered data. In general, the intensity of one or more bands may be determined using any of a variety of techniques, including known techniques. At a block 438, an intensity of a phosphate ν₁ band (nominally located at approximately 959 cm⁻¹) associated with the cartilage tissue is determined. Determining the intensity of this band may be performed in the same or similar manner as described with reference to block 434.

At a block 442, a ratio of the intensity determined at the block 434 with the intensity determined at the block 438 may be determined. Then, at a block 446, an indicator of osteoarthritis is determined based on the ratio determined at the block 446. It is believed that cartilage tissue affected by osteoarthritis has a higher carbonate/phosphate ratio as compared with cartilage not affected by osteoarthritis.

Determining the indicator may comprise determining in which of one or more sets of values the ratio falls by, for example, comparing the ratio to one or more thresholds. In one embodiment, the indicator may comprise an indication of whether or not osteoarthritis is present. In other embodiments, the indicator may comprise one of a plurality of levels indicating a probability or confidence level that osteoarthritis is present. In still other embodiments, the indicator may comprise one of a plurality of levels indicating a risk of developing ostearthritis. In yet other embodiments, the indicator may comprise one of a plurality of levels indicating a level of severity, a level of progression, etc., of osteoarthritis.

As described previously, the indicator determined at the block 446 may be based on additional factors such as one or more of an age of the patient, a weight of the patient, a prior weight of the patient, blood test data, synovial fluid test data, medical history data (e.g., past joint injuries), X-ray data, family history data, etc.

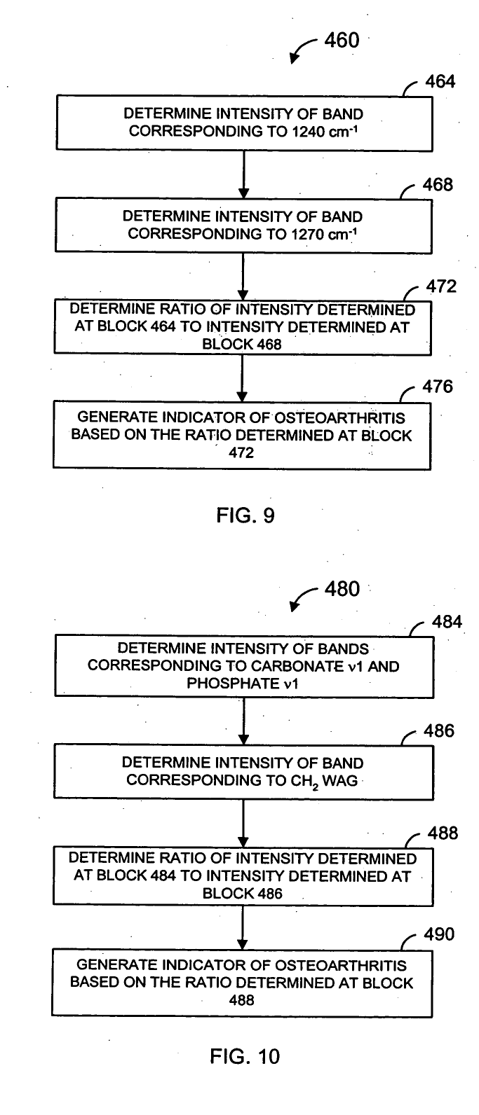

FIG. 9 is a flow diagram illustrating another embodiment of a method 460 for generating an indicator of osteoarthritis based on intensities of particular bands in a Raman spectrum of cartilage tissue. A similar technique may be employed for use with an IR spectrum of cartilage tissue.

At a block 464, an intensity of a band associated with the cartilage tissue having peak nominally at approximately 1240 cm⁻¹ is determined. Determining the intensity of this band may be performed in the same or similar manner as described above. At a block 468, an intensity of a band associated with the cartilage tissue having peak nominally at approximately 1270 cm⁻¹ is determined. Determining the intensity of this band may be performed in the same or similar manner as described above.

At a block 472, a ratio of the intensity determined at the block 464 with the intensity determined at the block 468 may be determined. Then, at a block 476, an indicator of osteoarthritis is determined based on the ratio determined at the block 472. It is believed that cartilage tissue affected by osteoarthritis has a higher 1240 cm⁻¹ band/1270 cm⁻¹ band ratio as compared with cartilage not affected by osteoarthritis.

Determining the indicator may comprise determining in which of one or more sets of values the ratio falls. In one embodiment, the indicator may comprise an indication of whether or not osteoarthritis is present. In other embodiments, the indicator may comprise one of a plurality of levels indicating a probability or confidence level that osteoarthritis is present. In still other embodiments, the indicator may comprise one of a plurality of levels indicating a risk of developing osteoarthritis. In yet other embodiments, the indicator may comprise one of a plurality of levels indicating a level of severity, a level of progression, etc., of osteoarthritis. As described previously, the indicator determined at the block 476 may be based on additional factors.

FIG. 10 is a flow diagram illustrating yet another embodiment of a method 480 for generating an indicator of a cartilage tissue condition based on intensities of particular bands in a Raman spectrum of cartilage tissue. A similar technique may be employed for use with an IR spectrum of cartilage tissue.

At a block 484, an intensity of one or more mineral bands associated with the cartilage tissue is determined. For example, the intensity of the carbonate ν₁ band and the phosphate ν₁ band may be determined by adding their individual intensities together. Determining the intensity of each individual band in the one or more mineral bands may be performed in the same or similar manner as described above. At a block 486, an intensity of a CH₂ wag band associated with the cartilage tissue having peak nominally at approximately 1446 cm⁻¹ is determined. Determining the intensity of this band may be performed in the same or similar manner as described above.

At a block 488, a ratio of the intensity determined at the block 484 with the intensity determined at the block 486 may be determined. Then, at a block 490, an indicator of osteoarthritis is determined based on the ratio determined at the block 488. It is believed that cartilage tissue affected by osteoarthritis has a lower mineral/CH₂ wag ratio as compared with cartilage not affected by osteoarthritis. With regard to the block 486, other bands associated with the collagen matrix of the cartilage tissue may be used in place of the CH₂ wag band such as amide I (1665 cm⁻¹), amide III (1240 cm⁻¹-1270 cm⁻¹), 855 cm⁻¹, 880 cm⁻¹, 919 cm⁻¹, etc. Generally, the ratio determined at the block 486 indicates the amount of mineral per collagen.

Determining the indicator may comprise determining in which of one or more sets of values the ratio falls. In one embodiment, the indicator may comprise an indication of whether or not osteoarthritis is present. In other embodiments, the indicator may comprise one of a plurality of levels indicating a probability or confidence level that osteoarthritis is present. In still other embodiments, the indicator may comprise one of a plurality of levels indicating a risk of developing osteoarthritis. In yet other embodiments, the indicator may comprise one of a plurality of levels indicating a level of severity, a level of progression, etc., of osteoarthritis. As described previously, the indicator determined at the block 490 may be based on additional factors.

In other embodiments, one or more of the methods described above with respect to FIGS. 8-10 may be combined. For example, an indicator of osteoarthritis could be determined based on the ratio determined at the block 442 of FIG. 8 and the ratio determined at the block 472 of FIG. 9. As another example, the indicator of osteoarthritis could be determined based on the ratio determined at the block 442 of FIG. 8 and the ratio determined at the block 488 of FIG. 10. As yet another example, the indicator of osteoarthritis could be determined based on the ratio determined at the block 472 of FIG. 9 and the ratio determined at the block 488 of FIG. 10. As still another example, the indicator of osteoarthritis could be determined based on the ratio determined at the block 442 of FIG. 8, the ratio determined at the block 472 of FIG. 9, and the ratio determined at the block 488 of FIG. 10. Multiple ratios may be used to determine an indicator of osteroarthritis using any of a variety of techniques. As one example, the multiple ratios may be mathematically combined and then the result could be compared to one or more thresholds. As another example, multiple indicators determined based on the multiple ratios could be mathematically combined.

Other information in the IR spectrum or the Raman spectrum of the cartilage tissue can be used in addition to, or as an alternative, the information described above. For example, information related to bands other than those described above could be used. Additionally, information related to the width, shape (e.g., whether or not a band has “shoulders”), height, etc. of particular bands could be used in determining a cartilage tissue condition. Additionally, more sophisticated analyses could be employed such as a cluster analysis, pattern matching, etc.

The locations of particular Raman bands described above with reference to FIGS. 8-10 were determined based on experiments with mice tissues. One of ordinary skill in the art will recognize that the locations of bands may vary based on, for example, testing error, age, species, etc. For instance, the locations may vary by up to plus or minus 3 cm⁻¹, or even more.

### EXPERIMENTS

In one experiment, Del 1 (+/−) transgenic mice containing 6 copies of a transgene with a small deletion mutation in the type II collagen gene and that were predisposed to early osteoarthritis were analyzed. The femoral articular cartilage was obtained from Del 1 (+/−) mice at 8 ages (2.5, 3, 5, 7, 9, 10, 13, and 16 months), with age-matched wildtype (wt) controls. The femoral articular cartilage was isolated en bloc and subject to Raman spectroscopy with 785 nm laser excitation and an Olympus BH-2 microscope equipped with a 20×/0.75 NA Zeiss Fluar objective. At each time point, the articular surfaces of three to four transects per femur were examined. Raw spectra were baselined with a polynomial, and curve fitted with GRAMS/AI © software. Bands associated with cartilage matrix proteins were analyzed using the Students t-test. All p values less than 0.05 were considered statistically significant.

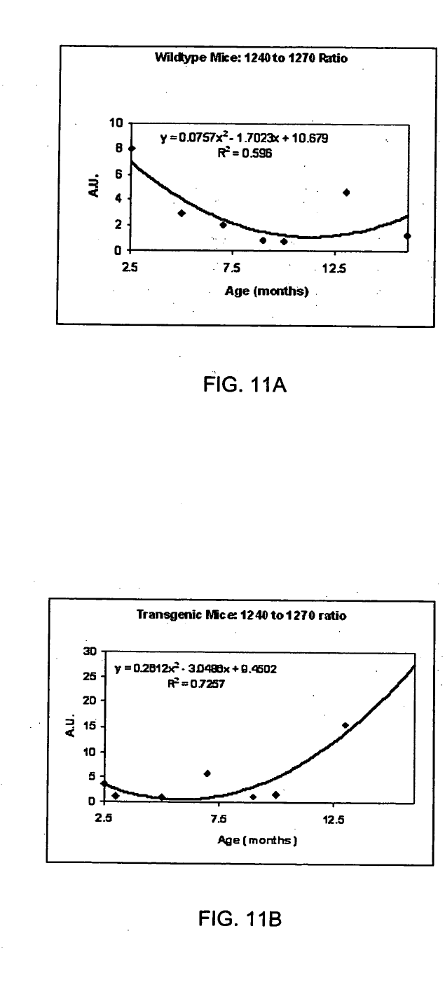

No statistically significant difference was observed between the 2.5 and 3 month old Del1 (+/−) and wt mice. At 5 months of age, however, some differences in the composition and structure of the tissue were detected between the Del 1 (+/I) and wt mice. In general, the Del 1 (+/−) mice had larger carbonate ν₁:phosphate ν₁ (CO₃:PO₄) ratios than wt mice, and this difference increased with age. The higher, CO₃: PO₄ ratio reflects a more carbonated mineral, which is more crystalline and has the potential to compensate for tissue weaknesses. In addition, the Del 1 (+/−) mice exhibited higher 1240 cm⁻¹:1270 cm⁻¹ band (1240:1270 band) area ratios than their age-matched controls, which indicates a more disordered secondary structure of collagen. The correlation between age and the 1240:1270 ratio was best fit with a second-degree polynomial. FIG. 11A is a graph showing 1240:1270 ratios of wt mice, the second-degree polynomial to which it was fit, and the R² value associated with the fit. FIG. 11B is a graph showing 1240:1270 ratios of Del 1 (+/−) mice, the second-degree polynomial to which it was fit, and the R² value associated with the fit. In both of FIGS. 11A and 11B, the vertical axes are in arbitrary units (A.U.).

Differences between Del 1 (+/−) and wt mice were discerned at as early as 5 months of age. The non-linear relationship between age and the 1240:1270 ratio suggests that at a particular age, the extracellular matrix accumulates changes in the tertiary structure, of the collagen fibrils, as is evidenced by the progressive decrease in overall order. This change occurred for the Del 1 (+/−) mice at approximately 6 months of age, and for the wt mice at approximately 11 months of age. It is possible that the 1240:1270 band area ratio, indicates the age at which irreversible damage begins to occur within the femoral articular cartilage.

In another experiment, Del 1 (+/−) transgenic mice containing 6 copies of a transgene with a small deletion mutation in the type II collagen gene and that were predisposed to early osteoarthritis were analyzed. Murine femoral articular cartilage was obtained from Del 1 (+/−) mice at 8 ages (2, 2.5, 3, 5, 7, 9, 13, and 16 months), with age-matched wildtype (wt) controls. The Del 1 (+/−) mice had early onset of flattening of femoral condyles, erosion of articular cartilage, sclerosis of subchondral bone, degeneration of the menisci, pyknotic chondrocyte nuclei, with clusters of reactive chondrocytes at the margins of the defects.

Raman spectra were obtained with 785 nm laser excitation. To improve signal-to-noise ratio images were acquired and component spectra were extracted using multivariate analysis allowing the separation of cartilage spectra from mineral spectra. Although there are similarities between the spectra of cartilage and bone matrix, the Raman spectra patterns are distinct because type II collagen is not chemically identical to type I collagen. Additionally, the Raman spectra information includes bands associated with specific proteoglycans in cartilage.

Differences between the Raman spectra of cartilage of Del 1 (+/−) and wt mice were observed. Also, differences in the Raman spectra of cartilage were observed with differences in age. Differences were particularly notable at the 1685 cm⁻¹ band, comparing the ages 13 months to 16 months, and comparing the ages 2 months and 7 months. It is believed that differences at the 1685 cm⁻¹ band possibly reflect immature crosslinks or ruptured crosslinks in collagen. At each point, the Del 1 (+/−) mice had a higher carbonate/phosphate ratio. This may indicate that Del 1 (+/−) mice cartilage had a more crystalline mineral content. Also, at each point, the Del 1 (+/−) mice had a higher 1240:1270 ratio. This may indicate that Del 1 (+/−) mice cartilage had a more disordered structure of collagen. Further, at each point, the Del 1 (+/−) mice had a lower mineral/matrix ratio, where the mineral/matrix ratio was calculated based on bands associated with carbonate and phosphate (mineral) and a band located at approximately 1446 cm−1 (matrix). This may indicate that Del 1 (+/−) mice cartilage had less mineral per collagen.

While the invention is susceptible to various modifications and alternative constructions, certain illustrative embodiments thereof have been shown in the drawings, and are described in detail herein. It should be understood, however, that there is no intention to limit the disclosure to the specific forms disclosed, but on the contrary, the intention is to cover all modifications, alternative constructions and equivalents falling within the spirit and scope of the disclosure as defined by the appended claims.

1. A method for evaluating a supporting tissue condition of a patient, the method comprising:
irradiating a portion of connective tissue of the patient using a light source;
receiving light from the portion of the connective tissue;
determining spectral content information associated with the received light; and
generating, based at least on the spectral content information, an indicator of the connective tissue condition.

2. A method as defined in claim 1, wherein generating the indicator of the connective tissue condition comprises generating an indicator of a bone tissue condition.

3. A method as defined in claim 1, wherein generating the indicator of the connective tissue condition comprises generating an indicator of a cartilage tissue condition.

4. A method as defined in claim 1, wherein irradiating the portion of connective tissue of the patient using the light source comprises irradiating the portion of connective tissue of the patient using a substantially monochromatic light source.

5. A method as defined in claim 4, wherein irradiating the portion of connective tissue of the patient using the substantially monochromatic light source comprises irradiating the portion of connective tissue of the patient using a substantially monochromatic light source that produces light having a wavelength substantially between 700 nanometers and 1100 nanometers.

6. A method as defined in claim 5, wherein irradiating the portion of connective tissue of the patient using the substantially monochromatic light source comprises irradiating the portion of connective tissue of the patient using a substantially monochromatic light source that produces light having a wavelength of substantially 785 nanometers.

7. A method as defined in claim 6, wherein irradiating the portion of connective tissue of the patient using the substantially monochromatic light source comprises irradiating the portion of connective tissue of the patient using a substantially monochromatic light source that produces light having a wavelength of substantially 830 nanometers.

8. A method as defined in claim 1, wherein irradiating the portion of connective tissue of the patient using the light source comprises irradiating the portion of connective tissue of the patient using an infrared light source.

9. A method as defined in claim 1, wherein irradiating the portion of connective tissue of the patient comprises at least one of irradiating the portion of connective tissue in vivo, irradiating the portion of the connective tissue through the skin of the patient, irradiating the portion of the connective tissue via an incision in the patient, and irradiating a biopsy of connective tissue removed from the patient.

10. A method as defined in claim 1, wherein receiving light from the portion of the connective tissue comprises at least one of receiving light scattered from the portion of the connective tissue, receiving light transmitted through the portion of the connective tissue, and receiving light reflected by the portion of the connective tissue.

11. A method as defined in claim 1, wherein determining spectral content information comprises at least one of determining Raman spectra and determining infrared spectra.

12. A method as defined in claim 1, wherein generating the indicator of the connective tissue condition comprises generating an indicator associated with at least one of osteoarthritis, rheumatoid arthritis, chondromalacia, polychondritis, relapsing polychondritis, a genetic disorder, and an acquired disorder.

13. A method as defined in claim 1, wherein the spectral content information includes a plurality of bands corresponding to received light at one or more wavelengths;
wherein generating the indicator of the connective tissue condition comprises determining at least one intensity of at least one band.

14. A method as defined in claim 13, wherein determining the at least one intensity of the at least one band comprises fitting a curve to at least one band.

15. A method as defined in claim 13, wherein determining the at least one intensity of the at least one band comprises calculating an area of at least one band.

16. A method as defined in claim 13, wherein determining the at least one intensity of the at least one band comprises calculating a height of at least one band.

17. A method as defined in claim 13, wherein generating the indicator of the connective tissue condition comprises:
determining a first intensity of at least a first band;
determining a second intensity of at least a second band; and
determining a first ratio of the first intensity and the second intensity.

18. A method as defined in claim 17, wherein generating the indicator of the connective tissue condition further comprises generating the indicator based at least in part on the first ratio.

19. A method as defined in claim 17, wherein the first band comprises a carbonate band and wherein the second band comprises a phosphate band.

20. A method as defined in claim 17, wherein the first band comprises a band associate and wherein the second band comprises a band at circa 1270 cm⁻¹.

21. A method as defined in claim 17, wherein determining the first intensity of the at least the first band comprises:
determining a third intensity of the first band;
determining a fourth intensity of a third band; and
generating the first intensity based on the third intensity and the fourth intensity.

22. A method as defined in claim 21, wherein determining the second intensity comprises determining an intensity of a band associated with a matrix of the connective tissue;
wherein determining the third intensity comprises determining an intensity of a carbonate band; and
wherein determining the fourth intensity comprises determining an intensity of a phosphate band.

23. A method as defined in claim 17, wherein generating the indicator of the connective tissue condition comprises:
determining a third intensity of at least a third band;
determining a fourth intensity of at least a fourth band;
determining a second ratio of the third intensity and the fourth intensity.

24. A method as defined in claim 23, wherein generating the indicator of the connective tissue condition further comprises wherein generating the indicator of the connective tissue condition based on the first ratio and the second ratio.

25. A method as defined in claim 1, further comprising determining whether the patient has the connective tissue condition based on the indicator of whether the patient has the connective tissue condition and on at least one of an age of the patient, height of the patient, a weight of the patient, prior weight history of the patient, a blood test, a synovial fluid test, a bone mineral density of the patient, an X-ray, prior medical history of the patient, and a family history of the patient.

26. An apparatus for evaluating a connective tissue condition of a patient, comprising:
a light source;
a light receiver to receive light from a portion of connective tissue of a patient irradiated by the light source;
a spectrum analyzer optically coupled to receive light received by the light receiver, the spectrum analyzer configured to generate spectral content information associated with the received light; and
a computing device communicatively coupled to the spectrum analyzer, the computing device configured to generate diagnostic information indicative of the connective tissue condition based at least in part on the spectral content information.

27. An apparatus as defined in claim 26, wherein the computing device is configured to generate diagnostic information indicative of a cartilage tissue condition.

28. An apparatus as defined in claim 26, wherein the computing device is configured to generate diagnostic information indicative of at least one of a bone tissue condition and a cartilage tissue condition.

29. An apparatus as defined in claim 26, wherein the light source comprises a substantially monochromatic light source.

30. An apparatus as defined in claim 29, wherein the light source produces light having a wavelength substantially between 700 nanometers and 1100 nanometers.

31. An apparatus as defined in claim 26, wherein the light source comprises an infrared light source.

32. An apparatus as defined in claim 26, wherein the light receiver comprises a microscope.

33. An apparatus as defined in claim 26, wherein the light receiver comprises an optical probe.

34. An apparatus as defined in claim 26, wherein the light receiver comprises a lens coupled to a needle.

35. An apparatus as defined in claim 34, wherein the light receiver further comprises at least one optical fiber coupled to the lens.

36. An apparatus as defined in claim 26, wherein the computing device comprises a digital circuit.

37. An apparatus as defined in claim 26, wherein the computing device comprises an analog circuit.

38. An apparatus as defined in claim 26, wherein the computing device comprises a mixed analog and digital circuit.

39. An apparatus as defined in claim 26, wherein the computing device comprises a processor coupled to a memory.

40. An apparatus as defined in claim 26, wherein the connective tissue condition comprises at least one of osteoarthritis, rheumatoid arthritis, chondromalacia, polychondritis, relapsing polychondritis, a genetic disorder, and an acquired disorder.

41. An apparatus as defined in claim 40, wherein the spectral content information includes a plurality of bands corresponding to received light at one or more wavelengths;
wherein the computing device is configured to determine at least one intensity of at least one band.

42. An apparatus as defined in claim 41, wherein the computing device is configured to fit a curve to the at least one band.

43. An apparatus as defined in claim 41, wherein the computing device is configured to determine an area of at least one band.

44. An apparatus as defined in claim 41, wherein the computing device is configured to determine a height of at least one band.

45. An apparatus as defined in claim 41, wherein the computing device is configured to determine a first intensity of at least a first band;
wherein the computing device is configured to determine a second intensity of at least a second band; and
wherein the computing device is configured to determine a first ratio of the first intensity and the second intensity.

46. An apparatus as defined in claim 26, wherein the computing device is configured to generate the diagnostic information indicative of the connective tissue condition further based on at least one of age of the patient, a height of the patient, a weight of the patient, a prior weight of the patient, blood test data, synovial fluid test data, a bone mineral density of the patient, data associated with an X-ray, prior medical history data, and family history data.

47. A method for evaluating a cartilage tissue condition of a patient, the method comprising:
irradiating a portion of cartilage tissue of the patient using a light source;
receiving light from the portion of the cartilage tissue;
determining Raman spectra information associated with the received light; and
generating, based at least on the Raman spectra information, an indicator of the cartilage tissue condition.

48. An apparatus for evaluating a cartilage tissue condition of a patient, comprising:
a light source;
a Raman probe to receive light scattered from a portion of cartilage tissue of a patient irradiated by the light source;
a spectrum analyzer coupled to receive light received by the light receiver and to determine Raman spectra information for the received light; and
a computing device coupled to the spectrum analyzer, the computing device configured to generate diagnostic information indicative of the cartilage tissue condition based at least in part on the Raman spectra information.
